(19) United States
(12) Patent Application Publication
(10) Pub. No.: US 2011/0129857 A1
(43) Pub. Date: Jun. 2, 2011

(54) Methods Of Detecting Or Monitoring Activity Of An Inflammatory Condition Or Neurodegenerative Condition

(75) Inventor: Ramesh C. Nayak, Tucson, AZ (US)

(73) Assignee: MSDX, INC., Tucson, AZ (US)

(21) Appl. No.: 12/954,505

(22) Filed: Nov. 24, 2010

Related U.S. Application Data
(60) Provisional application No. 61/264,760, filed on Nov. 27, 2009.
(60) Provisional application No. 61/371,122, filed on Aug. 5, 2010.
(60) Provisional application No. 61/393,254, filed on Oct. 14, 2010.

(51) Int. Cl. G01N 33/53; C07K 16/18

(52) U.S. Cl. 435/7.24; 436/501; 435/7.92; 435/7.1; 436/86; 530/350; 530/387.9

(57) ABSTRACT

An isolated aggrefatin protein elevated in multiple sclerosis patients as compared to healthy controls. Aggrefatin alone or in combination with other markers may be used as an indicator of an inflammatory condition and/or a neurodegenerative disease or condition such as multiple sclerosis, cancer, stroke, or other diseases. Aggrefatin alone or in combination with one or more other biomarkers may help monitor disease activity, detect a response to a therapy, or detect patient compliance with a therapy.

### CROSS REFERENCE

This application claims priority to U.S. provisional application Ser. No. 61/264,760 filed Nov. 27, 2009 and U.S. provisional application Ser. No. 61/371,122 filed Aug. 5, 2010, the specifications of which are incorporated herein by reference in their entirety.

### BACKGROUND OF THE INVENTION

Visfatin was originally identified as a putative cytokine that enhances the maturation of B cell precursors in the presence of Interleukin-7 (IL-7) and stem cell factor. It was first named “pre-B cell colony-enhancing factor” (PBEF). A gene encoding the bacterial nicotinamide phosphoribosyltransferase (nadV) was found to exhibit significant homology to the mammalian PBEF gene. It was demonstrated that the mouse PBEF gene conferred Nampt (nicotinamide phosphoribosyltransferase) enzymatic activity and NAD-independent growth to bacteria lacking nadV. The mouse PBEF gene encodes a Nampt enzyme capable of modulating intracellular NAD levels. The crystal structure of Nampt/PBEF/visfatin has been determined and they all show that this protein is a dimeric type II phosphoribosyl-transferase enzyme involved in NAD metabolism.

It has been reported that visfatin is enriched in the visceral fat of both humans and mice and that its plasma levels increase during the development of obesity. Visfatin is an adipokine, which is an adipocyte-derived cytokine. Adipokines may regulate metabolism and insulin resistance. Some adipokines, such as adiponectin and leptin, affect immune and inflammatory functions. It has been suggested that visfatin may affect insulin resistance by binding to the insulin receptor.

Visfatin is generally associated with obesity and metabolic syndrome, but visfatin is also considered to be a pro-inflammatory adipokine. It has been reported that recombinant visfatin activates human leukocytes and induces cytokine production. In CD14+ monocytes, visfatin induces the production of IL-1β, TNF-α, and especially IL-6. In vivo, visfatin induces circulating IL-6 in BALB/c mice. In patients with inflammatory bowel disease, plasma levels of visfatin are elevated and mRNA expression of visfatin is significantly increased in colonic tissue of Crohn's and ulcerative colitis patients compared with healthy controls. Macrophages, dendritic cells, and colonic epithelial cells might be additional sources of visfatin. Visfatin also leads to enhanced phagocytic activity.

It has been surprisingly discovered that the expression of a form of visfatin (e.g., a derivative, an aggregate, a complex), termed “aggrefatin,” is elevated in multiple sclerosis patients as compared to healthy controls. Aggrefatin alone or in combination with other markers may be useful as an indicator of an inflammatory condition and/or a neurodegenerative disease or condition such as multiple sclerosis, cancer, stroke, or other diseases. Aggrefatin alone or in combination with one or more other biomarkers may help monitor disease activity (e.g., relapse, remission, etc.). Monitoring disease activity may be useful for detecting a response (e.g., positive response, negative response, lack of response) to a therapy, for detecting patient compliance with a therapy, or for providing useful clinical information for disease management.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### SUMMARY OF THE INVENTION

The present invention features an isolated aggrefatin protein comprising several epitopes including conformational epitopes that are specifically produced as a consequence of aggregation. Furthermore, as a consequence of aggregation some epitopes of the native visfatin molecule are inaccessible and others are not. Accessible epitopes of the native sequence present in the aggregate include Phe-Lys-Asp-Pro-Val-Ala-Asp-Pro-Asn-Lys-Arg and other accessible epitopes exist in the sequences cywltnwietilvqswypitvatnsreq (aa 141-168 of Visfatin) and vtksysfdeirknaqlnieleaahh (as 467-491 of Visfatin). For example, in some embodiments, the isolated aggrefatin protein comprises an epitope of Phe-Lys-Asp-Pro-Val-Ala-Asp-Pro-Asn-Lys-Arg, and the isolated aggrefatin protein having a molecular weight between about 200 kDa to 800 kDa. In some embodiments, the isolated aggrefatin protein has quaternary structure. In some embodiments, the isolated aggrefatin protein is a homogeneous aggregate of visfatin. In some embodiments, the isolated aggrefatin protein is a heterogeneous aggregate of visfatin.

The isolated aggrefatin protein may be isolated in citrate, heparin, a fluoride (e.g., oxalate), a heparin derivative (e.g., low molecular weight heparin or Fondaparinux), a vitamin K agonist (e.g., Warfarin), an antithrombin activator, a direct thrombin inhibitor (e.g. argatroban, lepirudin, bivalirudin, dabigatran), a snake venom, a component such as Batroxobin, or a combination thereof. In some embodiments, when monitoring drug efficacy, the sample can be collected in the aforementioned tubes or optionally in other tubes.

The isolated aggrefatin protein may have increased stability when isolated in a medium comprising glycerol (e.g., between about 25 to 35% glycerol). Without wishing to limit the present invention to any theory or mechanism, it is believed that glycerol helps push the monomers and dimers of visfatin to form aggregates (e.g., by binding to existing aggregates or forming new ones, etc.). This may also be true for EDTA (or EGTA), which chelates divalent cations (e.g., magnesium, calcium) and optionally serum. The isolated aggrefatin protein may have increased stability when isolated in a medium comprising a chelating agent (e.g., ethylenediaminetetraacetic acid (EDTA), ethylene glycol tetraacetic acid (EGTA), etc.) The isolated aggrefatin protein may have increased stability when isolated in a medium comprising serum.

The present invention also features a kit comprising an antibody (e.g., a monoclonal antibody) specific for an isolated aggrefatin protein (the isolated aggrefatin protein comprising an epitope of Phe-Lys-Asp-Pro-Val-Ala-Asp-Pro-Asn-Lys-Arg and having a molecular weight between about 200 kDa to 800 kDa). In some embodiments, the antibody is raised to a sample obtained from a mammal (e.g., human, patient). In some embodiments, the sample is stored at less than about −20 degrees Celsius in a medium comprising ammonium sulfate.

The present invention also features a method of obtaining a measurement of an isolated aggrefatin protein. The method may comprise obtaining a sample (e.g., blood, cerebrospinal fluid, etc.) from a mammal (e.g., human, patient); storing the sample in a medium that lacks a divalent cation chelating agent (e.g., oxalate, citrate) or in a medium that has a divalent cation chelating agent that does not chelate as many divalent cations as ethylenediaminetetraacetid acid (EDTA) does (e.g., citrate, oxalate) or in a medium comprising either heparin, a heparin derivative, a vitamin K agonist, an antithrombin activator, a direct thrombin inhibitor, or a combination thereof; and detecting a level of aggrefatin expression in the sample. The method may further comprise storing the sample in a medium that comprises glycerol. In some embodiments, when monitoring drug efficacy, the sample can be collected in the aforementioned tubes or optionally in other tubes.

The present invention also features a method of modifying a sample obtained from a mammal (e.g., human, patient). The method may comprise subjecting the sample to either (i) a medium comprising glycerol (e.g., between about 25 to 35% glycerol); (ii) a medium that lacks a divalent cation chelating agent; (iii) a medium that has a divalent cation chelating agent that does not chelate as many divalent cations as ethylenediaminetetraacetid acid (EDTA) does (e.g., citrate, oxalate); (iv) a medium comprising either heparin, a heparin derivative, a vitamin K agonist, an antithrombin activator, a direct thrombin inhibitor, a snake venom, or a combination thereof; or (v) a medium comprising ammonium sulfate (e.g., between about 5 to 30% saturated ammonium sulfate).

The present invention also features a method of detecting a positive response to a therapy for a neurodegenerative disease or condition in a mammal. In some embodiments, the method comprises obtaining a first sample from the mammal and subjecting the first sample to a medium that lacks a divalent cation chelating agent or a medium that has a divalent cation chelating agent that does not chelate as many divalent cations as ethylenediaminetetraacetid acid (EDTA) does or to a medium comprising either heparin, a heparin derivative, a vitamin K agonist, an antithrombin activator, a direct thrombin inhibitor, or a combination thereof; administering the therapy to the mammal with the disease or condition; obtaining a second sample from the mammal and subjecting the second sample to a medium that lacks a divalent cation chelating agent or a medium that has a divalent cation chelating agent that does not chelate as many divalent cations as ethylenediaminetetraacetid acid (EDTA) does or to a medium comprising either heparin, a heparin derivative, a vitamin K agonist, an antithrombin activator, a direct thrombin inhibitor, or a combination thereof, the second sample is obtained a certain time period after the first sample; detecting a level of aggrefatin expression in both the first sample and the second sample; and comparing the level of aggrefatin expression in the first sample with a level of aggrefatin expression in the second sample, wherein a positive response to the therapy is detected if the level of the aggrefatin expression in the second sample is less than the level of the aggrefatin expression in the first sample.

In some embodiments, the therapy causes a change to a level of aggrefatin in the mammal. In some embodiments, the method further comprises obtaining a third sample from the mammal and subjecting the third sample to a medium that lacks a divalent cation chelating agent or a medium that has a divalent cation chelating agent that does not chelate as many divalent cations as ethylenediaminetetraacetid acid (EDTA) does or to a medium comprising either heparin, a heparin derivative, a vitamin K agonist, an antithrombin activator, a direct thrombin inhibitor, or a combination thereof, the third sample is obtained a certain time period after the second sample; detecting a level of aggrefatin expression the third sample; and comparing the level of aggrefatin expression in the third sample with either the level of aggrefatin in the first sample or the level of aggrefatin in the second sample, wherein a positive response to the therapy is detected if the level of the aggrefatin expression in the third sample is less than either the level of the aggrefatin expression in the first sample or the level of aggrefatin in the second sample. In some embodiments, the medium that lacks a divalent cation chelating agent or the medium that has a divalent cation chelating agent that does not chelate as many divalent cations as ethylenediaminetetraacetid acid (EDTA) is citrate or oxalate.

In some embodiments, the first sample or the second sample is a blood sample or cerebrospinal fluid sample. In some embodiments, detecting the level of aggrefatin expression comprises introducing an antibody to the sample, wherein the antibody binds to aggrefatin. In some embodiments, detecting the level of aggrefatin expression comprises subjecting the sample to a western blot, an enzyme-linked immunosorbent assay (ELISA), a lateral flow assay, an immunohistochemistry assay, a radioimmunoassay, a bioluminescent assay, a chemiluminescent assay, a mass spectrometry assay, a flow cytometry assay, a fluorescent assay, a calorimetric assay, a enzymatic assay, a ligand binding assay, or a combination thereof. In some embodiments, the method further comprises detecting a level of a second biomarker in the sample. In some embodiments, the second biomarker is perforin. In some embodiments, detecting a level of perforin in the sample comprises detecting a percentage of CD16+/perforin+ cells. In some embodiments, a positive response to the therapy is detected if the percentage of CD16+/perforin+ cells is more than about 75% of total CD16+ cells. In some embodiments, the second biomarker is myelin basic protein (MFB) or a HLA DR2 related allele.

The present invention also features a method of detecting a positive response to a therapy for a stroke in a mammal. In some embodiments, the method comprises obtaining a first sample from the mammal and subjecting the first sample to a medium that lacks a divalent cation chelating agent or a medium that has a divalent cation chelating agent that does not chelate as many divalent cations as ethylenediaminetetraacetid acid (EDTA) does; administering the therapy to the mammal with the stroke; obtaining a second sample from the mammal and subjecting the second sample to a medium that lacks a divalent cation chelating agent or a medium that has a divalent cation chelating agent that does not chelate as many divalent cations as ethylenediaminetetraacetid acid (EDTA) does, the second sample is obtained a certain time period after the first sample; detecting a level of aggrefatin expression in both the first sample and the second sample; and comparing the level of aggrefatin expression in the first sample with a level of aggrefatin expression in the second sample, wherein a positive response to the therapy is detected if the level of the aggrefatin expression in the second sample is less than the level of the aggrefatin expression in the first sample.

In some embodiments, the therapy causes a change to a level of aggrefatin in the mammal. In some embodiments, the medium that lacks a divalent cation chelating agent or the medium that has a divalent cation chelating agent that does not chelate as many divalent cations as ethylenediaminetetraacetid acid (EDTA) is citrate or oxalate. In some embodiments, the sample is a blood sample or cerebrospinal fluid sample. In some embodiments, detecting the level of aggrefatin expression comprises introducing an antibody to the sample, wherein the antibody binds to aggrefatin. In some embodiments, detecting the level of aggrefatin expression comprises subjecting the sample to a western blot, an enzyme-linked immunosorbent assay (ELISA), a lateral flow assay, an immunohistochemistry assay, a radioimmunoassay, a bioluminescent assay, a chemiluminescent assay, a mass spectrometry assay, a flow cytometry assay, a fluorescent assay, a colorimetric assay, a enzymatic assay, a ligand binding assay, or a combination thereof.

In some embodiments, the method further comprises detecting a level of a second biomarker in the sample. In some embodiments, the second biomarker is perforin, wherein detecting a level of perforin in the sample comprises detecting a percentage of CD16+/perforin+ cells. In some embodiments, a positive response to the therapy is detected if the percentage of CD16+/perforin+ cells is more than about 75% of total CD16+ cells. In some embodiments, the second biomarker is myelin basic protein (MPB) or a HLA DR2 related allele.

The present invention also features a method of detecting a neurodegenerative disease or condition in a mammal. In some embodiments, the method comprises obtaining a sample from the mammal; subjecting the sample to a medium that lacks a divalent cation chelating agent or a medium that has a divalent cation chelating agent that does not chelate as many divalent cations as ethylenediaminetetraacetid acid (EDTA) does or to a medium comprising either heparin, a heparin derivative, a vitamin K agonist, an antithrombin activator, a direct thrombin inhibitor, or a combination thereof; and detecting a level of aggrefatin expression in the sample; wherein the neurodegenerative disease or condition is determined if the level of aggrefatin expression in the sample is more than about two standard deviations higher than an average level of aggrefatin expression in a control sample.

In some embodiments, the control sample is subjected to a medium that lacks a divalent cation chelating agent or a medium that has a divalent cation chelating agent that does not chelate as many divalent cations as ethylenediaminetetraacetid acid (EDTA) does or to a medium comprising either heparin, a heparin derivative, a vitamin K agonist, an antithrombin activator, a direct thrombin inhibitor, or a combination thereof.

In some embodiments, the medium that lacks a divalent cation chelating agent or the medium that has a divalent cation chelating agent that does not chelate as many divalent cations as ethylenediaminetetraacetid acid (EDTA) is citrate or oxalate. In some embodiments, the sample is a blood sample or cerebrospinal fluid sample. In some embodiments, detecting the level of aggrefatin expression comprises introducing an antibody to the sample, wherein the antibody binds to aggrefatin. In some embodiments, detecting the level of aggrefatin expression comprises subjecting the sample to a western blot, an enzyme-linked immunosorbent assay (ELISA), a lateral flow assay, an immunohistochemistry assay, a radioimmunoassay, a bioluminescent assay, a chemiluminescent assay, a mass spectrometry assay, a flow cytometry assay, a fluorescent assay, a colorimetric assay, a enzymatic assay, a ligand binding assay, or a combination thereof. In some embodiments, method further comprises detecting a level of a second biomarker in the sample. In some embodiments, the second biomarker is perforin, wherein detecting a level of perforin in the sample comprises detecting a percentage of CD16+/perforin+ cells. In some embodiments, the neurodegenerative disease or condition is detected if the percentage of CD16+/perforin+ cells is less than about 75% of total CD16+ cells. In some embodiments, the second biomarker is myelin basic protein (MPB) or a HLA DR2 related allele. In some embodiments, method further comprises administering a treatment to the mammal if the level of aggrefatin expression in the sample is more than about two standard deviations higher than an average level of aggrefatin expression in a control sample.

The present invention also features a method of monitoring disease activity of a neurodegenerative disease or condition in a mammal. In some embodiments, the method comprises obtaining a first sample from the mammal and subjecting the first sample to a medium that lacks a divalent cation chelating agent or a medium that has a divalent cation chelating agent that does not chelate as many divalent cations as ethylenediaminetetraacetid acid (EDTA) does or to a medium comprising either heparin, a heparin derivative, a vitamin K agonist, an antithrombin activator, a direct thrombin inhibitor, or a combination thereof; obtaining a second sample from the mammal and subjecting the second sample to a medium that lacks a divalent cation chelating agent or a medium that has a divalent cation chelating agent that does not chelate as many divalent cations as ethylenediaminetetraacetid acid (EDTA) does or to a medium comprising either heparin, a heparin derivative, a vitamin K agonist, an antithrombin activator, a direct thrombin inhibitor, or a combination thereof; detecting a level of aggrefatin expression in both the first sample and the second sample; and comparing the level of aggrefatin expression in the first sample with a level of aggrefatin expression in the second sample. If the level of the aggrefatin expression in the second sample is more than the level of the aggrefatin expression in the first sample then the disease activity has increased; the level of the aggrefatin expression in the second sample is less than the level of the aggrefatin expression in the first sample then the disease activity has decreased; the level of the aggrefatin expression in the second sample is about the same as the level of the aggrefatin expression in the first sample then the disease activity has not increased or decreased.

The present invention also features a method of monitoring, detecting, or predicting a subsequent stroke in a mammal. In some embodiments, the method comprises obtaining a first sample from the mammal when the mammal has a stroke or after the mammal has had a stroke and subjecting the first sample to a medium that lacks a divalent cation chelating agent or a medium that has a divalent cation chelating agent that does not chelate as many divalent cations as ethylenediaminetetraacetid acid (EDTA) does or to a medium comprising either heparin, a heparin derivative, a vitamin K agonist, an antithrombin activator, a direct thrombin inhibitor, or a combination thereof; obtaining a second sample from the mammal and subjecting the second sample to a medium that lacks a divalent cation chelating agent or a medium that has a divalent cation chelating agent that does not chelate as many divalent cations as ethylenediaminetetraacetid acid (EDTA) does or to a medium comprising either heparin, a heparin derivative, a vitamin K agonist, an antithrombin activator, a direct thrombin inhibitor, or a combination thereof; detecting a level of aggrefatin expression in both the first sample and the second sample; and comparing the level of aggrefatin expression in the first sample with a level of aggrefatin expression in the second sample, wherein if the level of the aggrefatin expression in the second sample is more than the level of the aggrefatin expression in the first sample then the mammal is at high risk for a subsequent stroke.

In some embodiments, the medium that lacks a divalent cation chelating agent or the medium that has a divalent cation chelating agent that does not chelate as many divalent cations as ethylenediaminetetraacetid acid (EDTA) is citrate or oxalate. In some embodiments, the sample is a blood sample or cerebrospinal fluid sample. In some embodiments, detecting the level of aggrefatin expression comprises introducing an antibody to the sample, wherein the antibody binds to aggrefatin. In some embodiments, detecting the level of aggrefatin expression comprises subjecting the sample to a western blot, an enzyme-linked immunosorbent assay (ELISA), a lateral flow assay, an immunohistochemistry assay, a radioimmunoassay, a bioluminescent assay, a chemiluminescent assay, a mass spectrometry assay, a flow cytometry assay, a fluorescent assay, a colorimetric assay, a enzymatic assay, a ligand binding assay, or a combination thereof.

In some embodiments, the method further comprises detecting a level of a second biomarker in the sample. In some embodiments, the second biomarker is perforin, wherein detecting a level of perforin in the sample comprises detecting a percentage of CD16+/perforin+ cells. In some embodiments, the disease or condition is detected if the percentage of CD16+/perforin+ cells is less than about 75% of total CD16+ cells. In some embodiments, the second biomarker is myelin basic protein (MPB) or a HLA DR2 related allele.

The present invention also features a method of monitoring or detecting a neurodegenerative disease or condition in a mammal (e.g., human, patient). The method may comprise obtaining a sample (e.g., a blood sample, a CSF sample, etc.) from a mammal (e.g., a human, patient); subjecting the sample to a medium that lacks a divalent cation chelating agent or a medium that has a divalent cation chelating agent that does not chelate as many divalent cations as ethylenediaminetetraacetid acid (EDTA) does; and detecting a level of aggrefatin expression in the sample. The neurodegenerative disease or condition is determined if the level of aggrefatin expression in the sample is more than about 25 fold higher than an average level of aggrefatin expression in a control sample.

In some embodiments, the neurodegenerative disease or condition is determined if the level of aggrefatin expression in the sample is more than about 27 fold higher than an average level of aggrefatin expression in a control sample. In some embodiments, the neurodegenerative disease or condition is detected if the level of aggrefatin expression in the sample is more than about 50 fold higher than a level of aggrefatin expression in the control sample. In some embodiments, the neurodegenerative disease or condition is detected if the level of aggrefatin expression in the sample is more than about 58 fold higher than a level of aggrefatin expression in the control sample. In some embodiments, the neurodegenerative disease or condition is detected if the level of aggrefatin expression in the sample is more than about 75 fold higher than a level of aggrefatin expression in the control sample.

In some embodiments, detecting the level of aggrefatin expression comprises introducing an antibody to the sample, wherein the antibody binds to aggrefatin. In some embodiments, detecting the level of aggrefatin expression comprises subjecting the sample to a western blot, an enzyme-linked immunosorbent assay (ELISA), a lateral flow assay, an immunohistochemistry assay, a radioimmunoassay, a bioluminescent assay, a chemiluminescent assay, a mass spectrometry assay, a flow cytometry assay, a fluorescent assay, a colorimetric assay, a enzymatic assay, a ligand binding assay, or a combination thereof.

In some embodiments, the method further comprises detecting a level of a second biomarker in the sample. In some embodiments, the second biomarker is perforin, wherein detecting a level of perforin in the sample comprises detecting a percentage of CD16+/perforin+ cells. In some embodiments, the neurodegenerative disease or condition is detected if the level of aggrefatin expression in the sample is more than about 20 fold higher than a level of aggrefatin expression in the control sample and the percentage of CD16+/perforin+ cells is less than the total CD16+ cells, for example, less than about 75% of total CD16+ cells.

In some embodiments, the second biomarker is myelin basic protein (MPB). Detecting a level of MBP in the sample may comprise detecting a level of MBP expression. In some embodiments, detecting the level of MBP expression comprises introducing an antibody to the sample, wherein the antibody binds to MBP. In some embodiments, detecting the level of aggrefatin expression comprises subjecting the sample to a western blot, an enzyme-linked immunosorbent assay (ELISA), a lateral flow assay, an immunohistochemistry assay, a radioimmunoassay, a bioluminescent assay, a chemiluminescent assay, a mass spectrometry assay, a flow cytometry assay, a fluorescent assay, a colorimetric assay, a enzymatic assay, a ligand binding assay, or a combination thereof.

The second biomarker may be MBP (or another marker, e.g., HLA DR2 related allele or others, etc.). MBP may be detected in phagocytes (e.g., circulating phagocytes). In some embodiments, the second biomarker is a CNS protein in phagocytes.

In some embodiments, the method further comprises administering a treatment to the mammal if the level of aggrefatin expression in the sample is more than about 25 fold higher than an average level of aggrefatin expression in a control sample.

The present invention also features a method of detecting a positive response to a therapy for a neurodegenerative disease or condition. The method may comprise administering the therapy to a mammal (e.g., human, patient); obtaining a sample (e.g., a blood sample, a CSF sample, etc.) from the mammal (e.g., a human, patient) undergoing the therapy; subjecting the sample to a medium that lacks a divalent cation chelating agent or a medium that has a divalent cation chelating agent that does not chelate as many divalent cations as ethylenediaminetetraacetid acid (EDTA) does; and detecting a level of aggrefatin expression in the sample. A positive response to the therapy is detected if the level of aggrefatin expression in the sample is less than or equal to about 25 fold higher than an average level of aggrefatin expression in a control sample.

In some embodiments, a positive response to the therapy is detected if the level of aggrefatin expression in the sample is less than or equal to about 10 fold more than a level of aggrefatin expression in a control sample. In some embodiments, a positive response to the therapy is detected if the level of aggrefatin expression in the sample is less than or equal to about 2 fold more than a level of aggrefatin expression in a control sample. In some embodiments, a positive response to the therapy is detected if the level of aggrefatin expression in the sample is less than about a level of aggrefatin expression in a control sample.

The present invention also features a method of detecting a positive response to a therapy for a neurodegenerative disease or condition. The method may comprise administering the therapy to a mammal (e.g., human, patient) with the disease or condition; obtaining a sample (e.g., a blood sample, a CSF sample, etc.) from the mammal (e.g., a human, patient) with the disease or condition; subjecting the sample to a medium that lacks a divalent cation chelating agent or a medium that has a divalent cation chelating agent that does not chelate as many divalent cations as ethylenediaminetetraacetid acid (EDTA) does; detecting a level of aggrefatin expression in the sample; and comparing the level of aggrefatin expression in the first sample with a level of aggrefatin expression in a second sample, wherein the second sample is a second sample obtained from the mammal a certain time period before the first sample was obtained. If the level of the aggrefatin expression in the first sample is more than the level of the aggrefatin expression in the second sample then the level of the aggrefatin expression in the first sample is less than the level of the aggrefatin expression in the second sample then the disease activity has decreased; the level of the aggrefatin expression in the first sample is about the same as the level of the aggrefatin expression in the second sample then the disease activity has not increased or decreased.

In some embodiments, detecting the level of aggrefatin expression comprises introducing an antibody to the sample, wherein the antibody binds to aggrefatin. In some embodiments, detecting the level of aggrefatin expression comprises subjecting the sample to a western blot, an enzyme-linked immunosorbent assay (ELISA), a lateral flow assay, an immunohistochemistry assay, a radioimmunoassay, a bioluminescent assay, a chemiluminescent assay, a mass spectrometry assay, a flow cytometry assay, a fluorescent assay, a colorimetric assay, a enzymatic assay, a ligand binding assay, or a combination thereof.

In some embodiments, the method further comprises detecting a level of a second biomarker in the sample. In some embodiments, the second biomarker is perforin, wherein detecting a level of perforin in the sample comprises detecting a percentage of CD16+/perforin+ cells. In some embodiments, a positive response to the therapy is detected if the level of aggrefatin expression in the sample is less than or equal to about 20 fold more than a level of aggrefatin expression in a control sample and the percentage of CD16+/perforin+ cells is more than a certain percentage of total CH16+ cells, for example, about 75% of total CD16+ cells. In some embodiments, the second biomarker is myelin basic protein (MPB), wherein detecting a level of MBP in the sample comprises detecting a level of MBP expression. In some embodiments, detecting the level of MBP expression comprises introducing an antibody to the sample, wherein the antibody binds to MBP. In some embodiments, detecting the level of aggrefatin expression comprises subjecting the sample to a western blot, an enzyme-linked immunosorbent assay (ELISA), a lateral flow assay, an immunohistochemistry assay, a radioimmunoassay, a bioluminescent assay, a chemiluminescent assay, a mass spectrometry assay, a flow cytometry assay, a fluorescent assay, a calorimetric assay, a enzymatic assay, a ligand binding assay, or a combination thereof.

In some embodiments, the method is used to monitor disease activity of a disease or condition. In some embodiments, the method is used to monitor progression of the disease or condition. In some embodiments, the disease or condition is multiple sclerosis or a cancer or a stroke or type 2 diabetes.

The present invention also features a method of monitoring disease activity of a neurodegenerative disease or condition. The method may comprise obtaining a sample (e.g., a blood sample, a CSF sample, etc.) from a mammal (e.g., a human, patient) with the disease or condition; subjecting the sample to a medium that lacks a divalent cation chelating agent or a medium that has a divalent cation chelating agent that does not chelate as many divalent cations as ethylenediaminetetraacetid acid (EDTA) does; detecting a level of aggrefatin expression in the sample; and comparing the level of aggrefatin expression in the first sample with a level of aggrefatin expression in a second sample, wherein the second sample is a second sample obtained from the mammal a certain time period before the first sample was obtained. If the level of the aggrefatin expression in the first sample is more than the level of the aggrefatin expression in the second sample then the disease activity has increased; the level of the aggrefatin expression in the first sample is less than the level of the aggrefatin expression in the second sample then the disease activity has decreased; the level of the aggrefatin expression in the first sample is about the same as the level of the aggrefatin expression in the second sample then the disease activity has not increased or decreased.

In some embodiments, the method is used to determine a positive response, a negative response, or a lack of response to a therapy. In some embodiments, the method is used to detect an exacerbation of the neurodegenerative disease or condition prior to development of symptoms. In some embodiments, the method is used to detect a lack of patient compliance with a therapy. In some embodiments, the method is used for drug development.

The present invention also features a method of monitoring, detecting, or predicting a stroke in a mammal (e.g., human, patient). The method may comprise obtaining a sample (e.g., a blood sample, a CSF sample, etc.) from a mammal (e.g., a human, patient); subjecting the sample to a medium that lacks a divalent cation chelating agent or a medium that has a divalent cation chelating agent that does not chelate as many divalent cations as ethylenediaminetetraacetid acid (EDTA) does; and detecting a level of aggrefatin expression in the sample. A stroke condition is detected if the level of aggrefatin expression in the sample is more than about 20 fold higher than an average level of aggrefatin expression in a control sample. In some embodiments, a stroke condition is detected if the level of aggrefatin expression in the sample is more than about 12 fold higher than an average level of aggrefatin expression in a control sample. In some embodiments, a stroke condition is detected if the level of aggrefatin expression in the sample is more than about 25 fold higher than an average level of aggrefatin expression in a control sample. In some embodiments, a susceptibility to a future stroke is detected if the level of aggrefatin expression in the sample is more than about 10 fold higher than an average level of aggrefatin expression in a control sample. In some embodiments, an increased susceptibility to a future stroke is detected if the level of aggrefatin expression in the sample is more than about 20 fold higher than an average level of aggrefatin expression in a control sample. In some embodiments, an increased susceptibility to a future stroke is detected if the level of aggrefatin expression in the sample is more than about 30 fold higher than an average level of aggrefatin expression in a control sample. In some embodiments, an increased susceptibility to a future stroke is detected if the level of aggrefatin expression in the sample is more than about 40 fold higher than an average level of aggrefatin expression in a control sample. In some embodiments, an increased susceptibility to a future stroke is detected if the level of aggrefatin expression in the sample is more than about 50 fold higher than an average level of aggrefatin expression in a control sample.

In some embodiments, detecting the level of aggrefatin expression comprises introducing an antibody to the sample, wherein the antibody binds to aggrefatin. In some embodiments, detecting the level of aggrefatin expression comprises subjecting the sample to a western blot, an enzyme-linked immunosorbent assay (ELISA), a lateral flow assay, an immunohistochemistry assay, a radioimmunoassay, a bioluminescent assay, a chemiluminescent assay, a mass spectrometry assay, a flow cytometry assay, a fluorescent assay, a colorimetric assay, a enzymatic assay, a ligand binding assay, or a combination thereof. In some embodiments, the method further comprises detecting a level of a second biomarker in the sample. In some embodiments, the second biomarker is perforin, wherein detecting a level of perforin in the sample comprises detecting a percentage of CD16+/perforin+ cells. In some embodiments, the disease or condition is detected if the level of aggrefatin expression in the sample is more than about 20 fold higher than a level of aggrefatin expression in the control sample and the percentage of CD16+/perforin+ cells is less than the total CD16+ cells, for example, less than about 75% of total CD16+ cells. In some embodiments, the second biomarker is myelin basic protein (MFB), wherein detecting a level of MBP in the sample comprises detecting a level of MBP expression.

In some embodiments, detecting the level of MBP expression comprises introducing an antibody to the sample, wherein the antibody binds to MBP. In some embodiments, detecting the level of aggrefatin expression comprises subjecting the sample to a western blot, an enzyme-linked immunosorbent assay (ELISA), a lateral flow assay, an immunohistochemistry assay, a radioimmunoassay, a bioluminescent assay, a chemiluminescent assay, a mass spectrometry assay, a flow cytometry assay, a fluorescent assay, a colorimetric assay, a enzymatic assay, a ligand binding assay, or a combination thereof.

The present invention also features a method of detecting a positive response to a therapy for a stroke. The method may comprise administering the therapy to a mammal (e.g., human, patient); obtaining a sample (e.g., a blood sample, a CSF sample, etc.) from the mammal (e.g., a human, patient) undergoing the therapy; subjecting the sample to a medium that lacks a divalent cation chelating agent or a medium that has a divalent cation chelating agent that does not chelate as many divalent cations as ethylenediaminetetraacetid acid (EDTA) does; and detecting a level of aggrefatin expression in the sample. A positive response to the therapy is detected if the level of aggrefatin expression in the sample is less than or equal to about 20 fold more than a level of aggrefatin expression in a control sample.

In some embodiments, a positive response to the therapy is detected if the level of aggrefatin expression in the sample is less than or equal to about 25 fold more than a level of aggrefatin expression in a control sample. In some embodiments, a positive response to the therapy is detected if the level of aggrefatin expression in the sample is less than or equal to about 10 fold more than a level of aggrefatin expression in a control sample. In some embodiments, a positive response to the therapy is detected if the level of aggrefatin expression in the sample is less than or equal to about 2 fold more than a level of aggrefatin expression in a control sample. In some embodiments, a positive response to the therapy is detected if the level of aggrefatin expression in the sample is less than or equal to about a level of aggrefatin expression in a control sample.

In some embodiments, detecting the level of aggrefatin expression comprises introducing an antibody to the sample, wherein the antibody binds to aggrefatin. In some embodiments, detecting the level of aggrefatin expression comprises subjecting the sample to a western blot, an enzyme-linked immunosorbent assay (ELISA), a lateral flow assay, an immunohistochemistry assay, a radioimmunoassay, a bioluminescent assay, a chemiluminescent assay, a mass spectrometry assay, a flow cytometry assay, a fluorescent assay, a calorimetric assay, a enzymatic assay, a ligand binding assay, or a combination thereof.

In some embodiments, the method further comprises detecting a level of a second biomarker in the sample. In some embodiments, the second biomarker is perforin, wherein detecting a level of perforin in the sample comprises detecting a percentage of CD16+/perforin+ cells.

In some embodiments, a positive response to the therapy is detected if the level of aggrefatin expression in the sample is less than or equal to about 20 fold more than a level of aggrefatin expression in a control sample and the percentage of CD16+/perforin+ cells is more than a certain percentage of total CD16+ cells, for example about 75% of total CD16+ cells. In some embodiments, the second biomarker is myelin basic protein (MPB), wherein detecting a level of MBP in the sample comprises detecting a level of MBP expression. In some embodiments, detecting the level of MBP expression comprises introducing an antibody to the sample, wherein the antibody binds to MBP. In some embodiments, detecting the level of aggrefatin expression comprises subjecting the sample to a western blot, an enzyme-linked immunosorbent assay (ELISA), a lateral flow assay, an immunohistochemistry assay, a radioimmunoassay, a bioluminescent assay, a chemiluminescent assay, a mass spectrometry assay, a flow cytometry assay, a fluorescent assay, a calorimetric assay, a enzymatic assay, a ligand binding assay, or a combination thereof.

In some embodiments, the method is used to monitor disease activity of a disease or condition. In some embodiments, the method is used to monitor progression of the disease or condition.

The present invention also features a method of monitoring disease activity of a stroke. The method may comprise obtaining a sample (e.g., a blood sample, a CSF sample, etc.) from a mammal (e.g., a human, patient) with the disease or condition; subjecting the sample to a medium that lacks a divalent cation chelating agent or a medium that has a divalent cation chelating agent that does not chelate as many divalent cations as ethylenediaminetetraacetid acid (EDTA) does; and detecting a level of aggrefatin expression in the sample; comparing the level of aggrefatin expression in the first sample with a level of aggrefatin expression in a second sample, wherein the second sample is a second sample obtained from the mammal a certain time period before the first sample was obtained. If the level of the aggrefatin expression in the first sample is more than the level of the aggrefatin expression in the second sample then the disease activity has increased; the level of the aggrefatin expression in the first sample is less than the level of the aggrefatin expression in the second sample then the disease activity has decreased; the level of the aggrefatin expression in the first sample is about the same as the level of the aggrefatin expression in the second sample then the disease activity has not increased or decreased.

In some embodiments, the method is used to determine a positive response, a negative response, or a lack of response to a therapy. In some embodiments, the method is used to detect an exacerbation of the disease or condition prior to development of symptoms. In some embodiments, the method is used to detect a lack of patient compliance with a therapy. In some embodiments, the method is used for drug development.

### BRIEF DESCRIPTION OF THE DRAWINGS

FIG. 1 shows measurements of aggrefatin levels in plasma samples from 33 multiple sclerosis (MS) patients, 14 healthy subjects (C), and 17 stroke (S) patients.

FIG. 2 shows measurements of plasma aggrefatin and circulating CD16+/Perforin+ cells.

FIG. 3 shows measurements of neural antigen Myelin Basic Protein (MBP) and plasma aggrefatin levels (in peripheral blood mononuclear cells).

FIG. 4 is a schematic representation of an assay for aggrefatin.

FIG. 5 shows measurements of aggrefatin before and after treatment with Tysabri®.

FIG. 6 shows measurements of aggrefatin before and after treatment with Avonex®.

FIG. 7 shows measurements of aggrefatin before and after treatment with Copaxone®.

### DESCRIPTION OF PREFERRED EMBODIMENTS

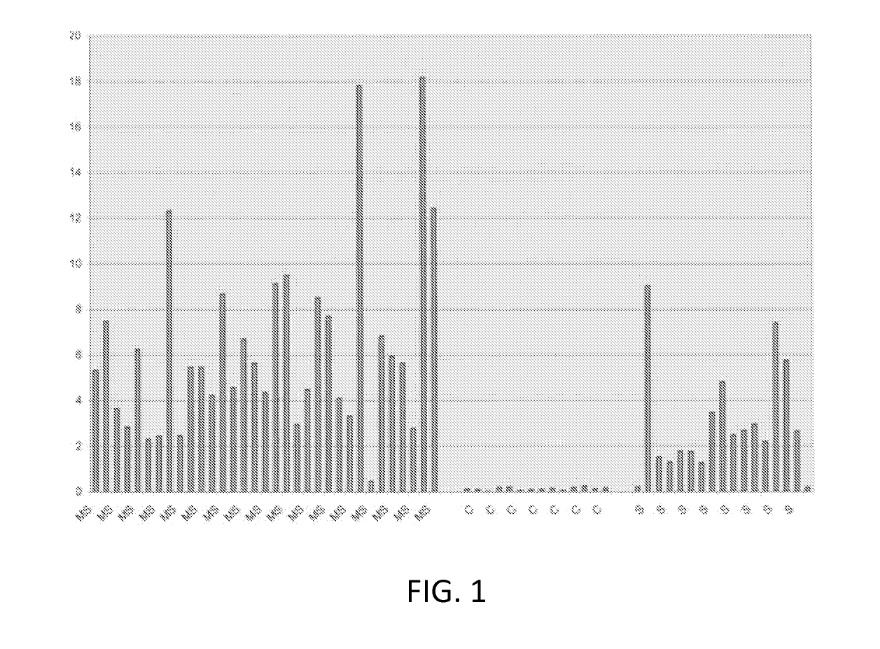

It has been surprisingly discovered that the expression of a form of visfatin (e.g., a derivative, an aggregate, a complex), termed “aggrefatin,” is elevated in multiple sclerosis (MS) patients as compared to healthy controls. FIG. 1 shows measurements of aggrefatin levels in plasma samples from 33 multiple sclerosis (MS) patients, 14 healthy subjects (C), and 17 stroke (S) patients. Aggrefatin levels in healthy controls (C) were very low, ranging from about 0.12-0.20 ng/mL. In comparison, MS patients had aggrefatin levels that were about 20-180 fold higher. Stroke patients had aggrefatin levels that were in the normal range or elevated, but generally not as greatly elevated as in MS. Aggrefatin may be a useful indicator of a disease or condition (e.g., an inflammatory condition, MS, a cancer, type 2 diabetes, etc.). In some embodiments, aggrefatin may be useful for monitoring disease activity (e.g., inflammatory status, relapse, remission, etc.) in MS and other diseases (e.g., cancer, stroke, type 2 diabetes, etc.).

Aggrefatin has an epitope of Phe-Lys-Asp-Pro-Val-Ala-Asp-Pro-Asn-Lys-Arg (FKDPVADPNKR), and glycerol (e.g., 30%) increases binding of antibodies that react with the FKDPVADPNKR epitope. Glycerol (e.g., 30%) decreases binding of antibodies that bind to other epitopes of aggrefatin. Aggrefatin may be a homogeneous aggregate of visfatin with a molecular weight greater than 200 kDa and up to 800 kDa by gel filtration chromatography on Sephacryl 3200. Aggrefatin may be a heterogeneous aggregate of Visfatin with itself and other proteins with a molecular weight (e.g., combined molecular weight) greater than 200 kilo Daltons and up to 800 kilo Daltons by gel filtration chromatography on Sephacryl S200. Other proteins and molecules that may form a heterogenous aggretate of visfatin may include but are not limited to fibrinogen, fibronectin, ferritin (e.g., light chain), NADH dehydrogenase subunit1, interferon induced transmembrane 3, etc.

Without wishing to limit the present invention to any theory or mechanism, it is believed that antibodies raised against visfatin peptides having amino acid sequence vtksysfdeirknaqlnieleaahh (aa 467-491) react well with aggrefatin immobilized on an ELISA. Antibodies raised against visfatin peptides having amino acid sequence cywltnwiet ilvqswypitvainsreq (aa 141-168) react well with aggrefatin immobilized on an ELISA but bind approximately half as well as antibodies reacting with vtksysfdeirknaqlnieleaahh (aa 467-491). Generally, the anti-Visfatin monoclonal antibody OMNI379 does not react with aggrefatin.

Generally, to monitor disease activity and for diagnostic use (alone or in combination with other markers), the sample is collected in a citrate anticoagulation tube or a fluoride (oxalate) anticoagulation tube or a heparin anticoagulation tube. Blood may also be collected into tubes using other anticoagulants that do not affect divalent cation (e.g., calcium) levels, e.g., heparin derivatives such as low molecular weight heparin or Fondaparinux, Vitamin K agonists (Warfarin), antithrombin activators, direct thrombin inhibitors (e.g. argatroban, lepirudin, bivalirudin, and dabigatran) and snake venoms and components such as Batroxobin. In some embodiments, when monitoring drug efficacy, the sample can be collected in the aforementioned tubes or optionally in other tubes. For example, without wishing to limit the present invention to any theory or mechanism, it is believed that for diagnostic purposes and/or for disease monitoring purposes, sample collection in EDTA does not produce a reliable and/or reproducible control. Thus, for diagnostic purposes and/or for disease monitoring purposes, samples are collected in a medium that lacks a divalent cation chelating agent or a medium that has a divalent cation chelating agent that does not chelate as many divalent cations as ethylenediaminetetraacetid acid (EDTA) does. For monitoring drug efficacy, however, EDTA medium may be used (or other mediums).

Subjecting samples to a medium that does chelate divalent cations (e.g., calcium, magnesium) strongly, for example EDTA, may be used to measure total (aggregated and non-aggregated) visfatin. For example, without wishing to limit the present invention to any theory or mechanism, it is believed that subjecting the sample to such a chelator (e.g., EDTA) forces the visfatin molecules to aggregate.

Aggrefatin in combination with one or more other biomarkers may help detect a disease or condition (e.g., an inflammatory condition, a neurodegenerative disease or condition, multiple sclerosis, a cancer, a stroke, type 2 diabetes, etc.) or monitor disease activity (e.g., inflammatory status, relapse, remission, immunoregulatory status, active demyelination, active neurodegeneration, drug compliance, etc.). Aggrefatin may also be used in combination with biomarkers for genetic predispositions and/or trigger events. Examples of biomarkers for genetic predispositions may include but are not limited to: interferon regulatory factor (IRF) 7, HLA-class I markers, such as but not limited to HLA-A*02, HLA-Cw*05 and MOG-142L; MS-associated kinesin motor protein KIF1B; rs17368528 SNP in the fifth exon of the hexose-6-phosphate dehydrogenase (H6PD) gene; HLA-class II markers such as DRB1*03, DRB1*1501, DRB1*03011, and DQB1*0602, The haplotypes DRB1*1501, DQA1*0102, DQB1*0602, DRB1*1303, DQA1*05, and DQB1*030; and TNFa9 allele and the biallelic combination (CCR5d32,DRB1*04). Examples of biomarkers for trigger events may include but are not limited to: dihydroxy vitamin D3 levels, Epstein-Barr Virus nuclear antigen and antibodies to same, phosphorylated dihydroxy ceramides, human endogenous retrovirus (HERV) genes, proteins and antibodies.

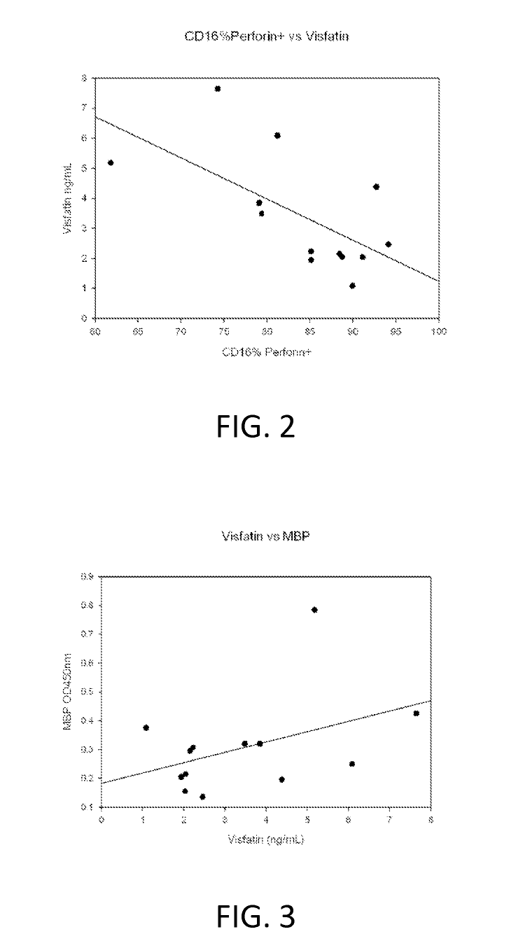

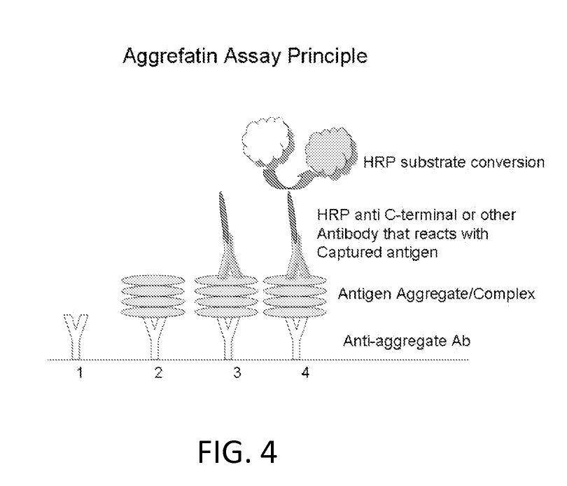

FIG. 2 shows a correlation between plasma aggrefatin and circulating CD16+/Perforin+ cells. It has been surprisingly discovered that the circulating level of CD16+/Perforin+ cells correlates with remission/therapeutic efficacy in MS. High levels of CD16+/Perforin+cells are associated with low levels of disease activity. It has also been surprisingly discovered that high levels of CD16+/Perforin+ cells correlate with low levels of plasma aggrefatin, suggesting that CD16÷/Perforin+ cell levels correlate inversely with the inflammatory status of the patient and therefore both measurements may be used as indicators of the level of disease activity. This may help physician, for example, determine whether a treatment (e.g., an anti-inflammatory treatment) should be administered, whether a treatment is functioning to reduce or maintain disease activity levels, or whether a treatment is failing to reduce or maintain disease activity levels.

FIG. 3 shows a correlation between the neural antigen Myelin Basic Protein (MBP) and plasma aggrefatin levels (in peripheral blood mononuclear cells). Circulating cells carry neural antigens (e.g., MBP) as cargo in neurological diseases. It has been surprisingly discovered that increased levels of MBP in circulating cells correlates with active inflammatory demyelination and increased aggrefatin levels. The measurement of MBP and aggrefatin may help indicate the presence of active inflammatory demyelination. Aggrefatin may also be compared to other markers, such as Tau and/or hippocalcin1-like 1. This may provide a physician with clinical information that will aid in determining disease management strategy.

As described above, aggrefatin alone or in combination with other markers may provide clinically useful information not just for disease or condition detection (e.g., an inflammatory condition, a neurodegenerative condition or disease, a stroke, multiple sclerosis, a cancer, etc.) but also for disease management (e.g., a means of monitoring disease activity, for example inflammatory status, relapse, remission, etc. in MS and other diseases).

For example, monitoring disease activity via aggrefatin (alone or in combination with other markers) may help determine whether or not a therapy is effective (e.g., detecting a positive, a negative response, or a lack of response to a therapy). In some embodiments, monitoring disease activity via aggrefatin (alone or in combination with other markers) may help detect an exacerbation of the disease or condition prior to the development of symptoms. In some embodiments, monitoring disease activity via aggrefatin (alone or in combination with other markers) may help detect a lack of patient compliance with a therapy. In some embodiments, monitoring disease activity via aggrefatin (alone or in combination with other markers) may help drug development studies.

In some embodiments, a disease or condition may be detected by detecting aggrefatin levels (and/or levels of other markers) in combination with performing other tests (e.g., magnetic resonance imaging (MRI)).

In some embodiments, aggrefatin is measured throughout a time course. For example, aggrefatin is measured at Time₁ and again at Time₂, wherein a certain time frame has elapsed in between Time₁ and Time₂. In some embodiments, a drug or treatment/therapy is administered to the individual (e.g., mammal, patient, research animal, etc.) after Time₁. In some embodiments, a drug or treatment/therapy is administered to the individual (e.g., mammal, patient, research animal, etc.) at Time₁ (or around Time₁, for example right before, right after, etc.). The time course may be used to determine, for example, a positive, negative, or lack of response to a treatment or therapy.

In some embodiments, the time frame in between Time₁ and Time₂ is between about 30 minutes to 1 hour. In some embodiments, the time frame in between Time₁ and Time₂ is between about 1 to 2 hours. In some embodiments, the time frame in between Time₁ and Time₂ is between about 2 to 12 hours. In some embodiments, the time frame in between Time₁ and Time₂ is between about 12 to 24 hours. In some embodiments, the time frame in between Time₁ and Time₂ is between about 24 to 72 hours. In some embodiments, the time frame in between Time₁ and Time₂ is between about 3 to 5 days. In some embodiments, the time frame in between Time₁ and Time₂ is between about 5 to 10 days. In some embodiments, the time frame in between Time₁ and Time₂ is between about 10 to 14 days. In some embodiments, the time frame in between Time₁ and Time₂ is between about 2 to 4 weeks. In some embodiments, the time frame in between Time₁ and Time₂ is between about 4 to 6 weeks. In some embodiments, the time frame in between Time and Time₂ is between about 6 to 10 weeks. In some embodiments, the time frame in between Time₁ and Time₂ is between about 2 to 4 months. In some embodiments, the time frame in between Time₁ and Time₂ is between about 4 to 6 months. In some embodiments, the time frame in between Time₁ and Time₂ is between about 6 to 12 months. In some embodiments, the time frame in between Time₁ and Time₂ is between about 1 to 2 years. In some embodiments, the time frame in between Time₁ and Time₂ is between about 2 to 4 years. In some embodiments, the time frame in between Time₁ and Time₂ is more than about 4 years.

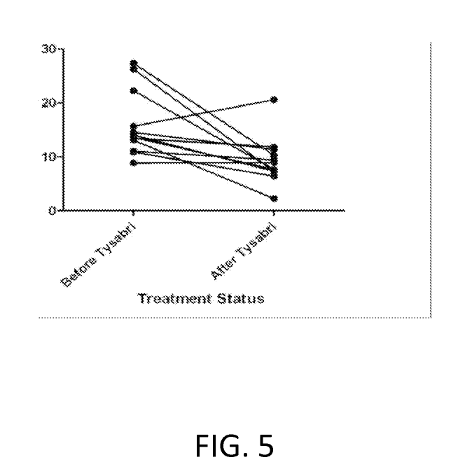

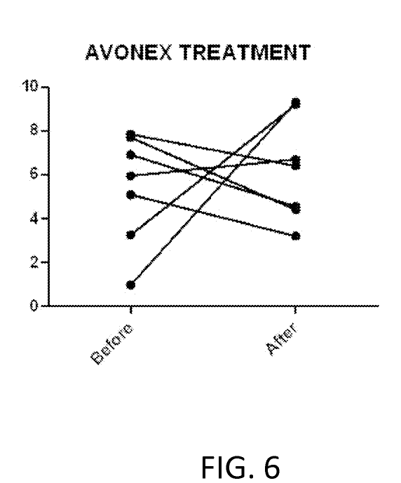

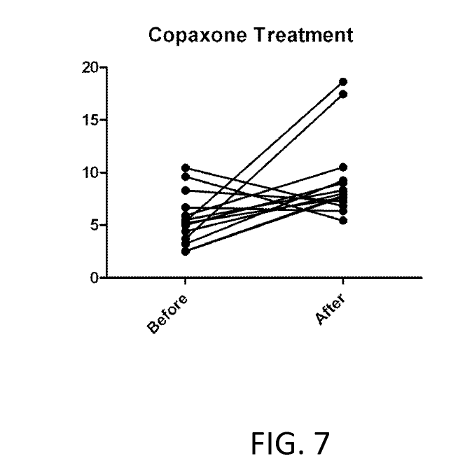

FIG. 5 shows measurements of aggrefatin before and after treatment with Tysabri®. Ten of twelve patients showed a decrease in aggrefatin levels after treatment. FIG. 6 measurements of aggrefatin before and after treatment with Avonex®. Four of seven patients showed a decrease in aggrefatin levels after treatment. FIG. 7 measurements of aggrefatin before and after treatment with Copaxone®. Four of fourteen patients showed a decrease in aggrefatin levels after treatment.

The present invention features methods of monitoring or detecting a neurodegenerative disease or condition (e.g., multiple sclerosis). In some embodiments, the method comprises obtaining a sample (e.g., blood sample or derivative thereof, cerebrospinal fluid (CSF), etc.) from a mammal (e.g., a human, patient) and detecting a level of aggrefatin expression in the sample. The neurodegenerative disease or condition (e.g., multiple sclerosis) may be determined if the level of aggrefatin expression in the sample is more than about 5 fold higher than an average level of aggrefatin expression in a control sample. The neurodegenerative disease or condition (e.g., multiple sclerosis) may be determined if the level of aggrefatin expression in the sample is more than about 10 fold higher than an average level of aggrefatin expression in a control sample. The neurodegenerative disease or condition (e.g., multiple sclerosis) may be determined if the level of aggrefatin expression in the sample is more than about 20 fold higher than an average level of aggrefatin expression in a control sample. The neurodegenerative disease or condition (e.g., multiple sclerosis) may be determined if the level of aggrefatin expression in the sample is more than about 25 fold higher than an average level of aggrefatin expression in a control sample. The neurodegenerative disease or condition (e.g., multiple sclerosis) may be determined if the level of aggrefatin expression in the sample is more than about 27 fold higher than an average level of aggrefatin expression in a control sample. The neurodegenerative disease or condition (e.g., multiple sclerosis) may be determined if the level of aggrefatin expression in the sample is more than about 50 fold higher than an average level of aggrefatin expression in a control sample. The neurodegenerative disease or condition (e.g., multiple sclerosis) may be determined if the level of aggrefatin expression in the sample is more than about 28 fold higher than an average level of aggrefatin expression in a control sample. The neurodegenerative disease or condition (e.g., multiple sclerosis) may be determined if the level of aggrefatin expression in the sample is more than about 75 fold higher than an average level of aggrefatin expression in a control sample.

In some embodiments, detecting the level of aggrefatin expression comprises introducing an antibody to the sample, wherein the antibody binds to aggrefatin. However, the present invention is not limited to detecting the level of aggrefatin expression with an antibody. In some embodiments, detecting the level of aggrefatin expression comprises subjecting the sample to a western blot, an enzyme-linked immunosorbent assay (ELISA), a lateral flow assay, an immunohistochemistry assay, a radioimmunoassay, a bioluminescent assay, a chemiluminescent assay, a mass spectrometry assay, a flow cytometry assay, a fluorescent assay, a colorimetric assay, a enzymatic assay, a ligand binding assay, or a combination thereof.

The level of aggrefatin may be detected in combination with a second (or more) biomarker, for example perforin and/or myelin basic protein (MBP) and/or another biomarker. Detecting perforin may comprise detecting a percentage of CD16+/perforin+ cells. In some embodiments, the disease or condition is detected if the level of aggrefatin expression in the sample is more than about 20 fold higher than a level of aggrefatin expression in the control sample and the percentage of CD16+/perforin+ cells is less than the total CD16+ cells, for example, less than about 75% of total CD16+ cells. Detecting MBP may comprise detecting a level of MBP in the sample comprises detecting a level of MBP expression, for example introducing an antibody to the sample, wherein the antibody binds to MBP.

The present invention also features methods of detecting a positive response to a therapy for a neurodegenerative disease or condition (e.g., multiple sclerosis). The method may comprise obtaining a sample (e.g., blood sample or derivative thereof, cerebrospinal fluid (CSF), etc.) from a mammal (e.g., human, patient) undergoing the therapy and detecting a level of aggrefatin expression in the sample. The methods may be used to monitor disease activity of a disease or condition or monitor progression of a disease or condition.

A positive response to the therapy may be detected if the level of aggrefatin expression in the sample is less than or equal to about 25 fold higher than an average level of aggrefatin expression in a control sample. A positive response to the therapy may be detected if the level of aggrefatin expression in the sample is less than or equal to about 10 fold more than a level of aggrefatin expression in a control sample. A positive response to the therapy may be detected if the level of aggrefatin expression in the sample is less than or equal to about 2 fold more than a level of aggrefatin expression in a control sample. A positive response to the therapy may be detected if the level of aggrefatin expression in the sample is less than about a level of aggrefatin expression in a control sample.

The method may further comprise comparing the level of aggrefatin expression in the first sample with a level of aggrefatin expression in a second sample, wherein the second sample is obtained from the mammal a certain time period before the first sample is obtained. In some embodiments, if the level of the aggrefatin expression in the first sample is more than the level of the aggrefatin expression in the second sample then the disease activity has increased; if the level of the aggrefatin expression in the first sample is less than the level of the aggrefatin expression in the second sample then the disease activity has decreased; and/or if the level of the aggrefatin expression in the first sample is about the same as the level of the aggrefatin expression in the second sample then the disease activity has not increased or decreased.

Detecting the level of aggrefatin expression may comprise introducing an antibody to the sample, wherein the antibody binds to aggrefatin. In some embodiments, detecting the level of aggrefatin may comprise subjecting the sample to a western blot, an enzyme-linked immunosorbent assay (ELISA), a lateral flow assay, an immunohistochemistry assay, a radioimmunoassay, a bioluminescent assay, a chemiluminescent assay, a mass spectrometry assay, a flow cytometry assay, a fluorescent assay, a colorimetric assay, a enzymatic assay, a ligand binding assay, or a combination thereof.

The level of aggrefatin may be detected in combination with a second (or more) biomarker, for example perforin and/or myelin basic protein (MBP) and/or another biomarker. Detecting perforin may comprise detecting a percentage of CD16+/perforin+ cells. In some embodiments, a positive response to the therapy is detected if the level of aggrefatin expression in the sample is less than or equal to about 20 fold more than a level of aggrefatin expression in a control sample and the percentage of CD16+/perforin+ cells is more than a certain percentage of total CH16+ cells, for example, about 75% of total CD16+ cells. Detecting MBP may comprise detecting a level of MBP in the sample comprises detecting a level of MBP expression, for example introducing an antibody to the sample, wherein the antibody binds to MBP.

The present invention also features methods of monitoring disease activity (e.g., of multiple sclerosis). The method may comprise obtaining a sample (e.g., blood sample or derivative thereof, cerebrospinal fluid (CSF), etc.) from a mammal (e.g., human, patient) with the disease or condition, detecting a level of aggrefatin expression in the sample, and comparing the level of aggrefatin expression in the first sample with a level of aggrefatin expression in a second sample. The second sample may be a sample obtained from the mammal a certain time period before the first sample is obtained. In some embodiments, if (i) the level of the aggrefatin expression in the first sample is more than the level of the aggrefatin expression in the second sample then the disease activity has increased; (ii) the level of the aggrefatin expression in the first sample is less than the level of the aggrefatin expression in the second sample then the disease activity has decreased; and/or (iii) the level of the aggrefatin expression in the first sample is about the same as the level of the aggrefatin expression in the second sample then the disease activity has not increased or decreased. The methods may be used to determine a positive response, a negative response, or a lack of response to a therapy; to detect an exacerbation of the disease or condition prior to development of symptoms; to detect a lack of patient compliance with a therapy; and/or for drug development.

The present invention also features a method of monitoring, detecting, or predicting a stroke in a mammal (e.g., human, patient). The method may comprise obtaining a sample (e.g., a blood sample or derivative thereof, cerebrospinal fluid (CSF), etc.) from a mammal (e.g., a human, patient) and detecting a level of aggrefatin expression in the sample. A stroke condition may be detected if the level of aggrefatin expression in the sample is more than about 12 fold higher than an average level of aggrefatin expression in a control sample. A stroke condition may be detected if the level of aggrefatin expression in the sample is more than about 20 fold higher than an average level of aggrefatin expression in a control sample. A stroke condition may be detected if the level of aggrefatin expression in the sample is more than about 25 fold higher than an average level of aggrefatin expression in a control sample.

A susceptibility to a future stroke may be detected if the level of aggrefatin expression in the sample is more than about 10 fold higher than an average level of aggrefatin expression in a control sample. A susceptibility to a future stroke may be detected if the level of aggrefatin expression in the sample is more than about 20 fold higher than an average level of aggrefatin expression in a control sample. A susceptibility to a future stroke may be detected if the level of aggrefatin expression in the sample is more than about 30 fold higher than an average level of aggrefatin expression in a control sample. A susceptibility to a future stroke may be detected if the level of aggrefatin expression in the sample is more than about 40 fold higher than an average level of aggrefatin expression in a control sample. A susceptibility to a future stroke may be detected if the level of aggrefatin expression in the sample is more than about 50 fold higher than an average level of aggrefatin expression in a control sample.

Detecting the level of aggrefatin expression may comprise introducing an antibody to the sample, wherein the antibody binds to aggrefatin. Detecting the level of aggrefatin expression may comprise subjecting the sample to a western blot, an enzyme-linked immunosorbent assay (ELISA), a lateral flow assay, an immunohistochemistry assay, a radioimmunoassay, a bioluminescent assay, a chemiluminescent assay, a mass spectrometry assay, a flow cytometry assay, a fluorescent assay, a calorimetric assay, a enzymatic assay, a ligand binding assay, or a combination thereof.

The present invention also features a method of detecting a positive response to a therapy for a stroke. The method may comprise obtaining a sample (e.g., a blood sample or derivative thereof, cerebrospinal fluid (CSF), etc.) from a mammal (e.g., a human, patient) undergoing the therapy and detecting a level of aggrefatin expression in the sample. A positive response to the therapy may be detected if the level of aggrefatin expression in the sample is less than or equal to about 20 fold more than a level of aggrefatin expression in a control sample. A positive response to the therapy may be detected if the level of aggrefatin expression in the sample is less than or equal to about 25 fold more than a level of aggrefatin expression in a control sample. A positive response to the therapy may be detected if the level of aggrefatin expression in the sample is less than or equal to about 10 fold more than a level of aggrefatin expression in a control sample. A positive response to the therapy may be detected if the level of aggrefatin expression in the sample is less than or equal to about 2 fold more than a level of aggrefatin expression in a control sample. A positive response to the therapy may be detected if the level of aggrefatin expression in the sample is less than or equal to about a level of aggrefatin expression in a control sample. In some embodiments, the method is used to monitor disease activity of a disease or condition. In some embodiments, the method is used to monitor progression of the disease or condition.

The present invention also features methods of monitoring disease activity of a stroke. In some embodiments, the methods comprise obtaining a sample (e.g., blood sample or derivative thereof, cerebrospinal fluid (CSF), etc.) from a mammal (e.g., human, patient) with the disease or condition, detecting a level of aggrefatin expression in the sample, and comparing the level of aggrefatin expression in the first sample with a level of aggrefatin expression in a second sample. The second sample is a sample obtained from the mammal a certain time period before the first sample is obtained. In some embodiments, if (i) the level of the aggrefatin expression in the first sample is more than the level of the aggrefatin expression in the second sample then the disease activity has increased; (ii) the level of the aggrefatin expression in the first sample is less than the level of the aggrefatin expression in the second sample then the disease activity has decreased; (iii) the level of the aggrefatin expression in the first sample is about the same as the level of the aggrefatin expression in the second sample then the disease activity has not increased or decreased. The methods may be used to determine a positive response, a negative response, or a lack of response to a therapy; to detect an exacerbation of the disease or condition prior to development of symptoms; to detect a lack of patient compliance with a therapy; and/or for drug development.

The present invention also features the isolated aggrefatin protein. The aggrefatin protein comprises an epitope of Phe-Lys-Asp-Pro-Val-Ala-Asp-Pro-Asn-Lys-Arg. Generally, the isolated aggrefatin protein has a molecular weight greater than 200 kDa (e.g., a molecular weight between about 200 kDa to 800 kDa). The isolated aggrefatin protein may have quaternary structure (e.g., multiple peptides, multiple subunits). The isolated aggrefatin protein may be a homogeneous aggregate of visfatin (e.g., multiple visfatin molecules, visfatin fragments, or visfatin-like molecules). The isolated aggrefatin protein may be a heterogeneous aggregate of visfatin (e.g., a complex with visfatin, or a fragment thereof or a visfatin-like molecule) and a different non-visfatin molecule).

Citrate or heparin (e.g., naturally occurring, commercially made, derivates of heparin, etc.) or other materials (e.g., oxalate) may be used to isolate the aggrefatin protein. Without wishing to limit the present invention to any theory or mechanism, it is believed that citrate and heparin chelate divalent cations (e.g., calcium, magnesium) less than ethylenediaminetetreacetic acid (EDTA). Or said differently, EDTA chelates divalent cations better than heparin and citrate do. The present invention is not limited to isolation of the aggrefatin protein in citrate or heparin. In some embodiments, the aggrefatin protein is isolated in ammonium sulfate (e.g., between about 5 to 10% saturated ammonium sulfate, between about 10 to 15% saturated ammonium sulfate, between about 15 to 20% saturated ammonium sulfate, between about 20 to 30% saturated ammonium sulfate. For example, the molecule may be “salted out” with ammonium sulfate.

In some embodiments, the aggrefatin protein is stored at a temperature less than 0 degrees Celsius (e.g., −20 degrees Celsius, between about 0 to −10 degrees Celsius, between about −10 to −20 degrees Celsius, between about −15 to −25 degrees Celsius, between about −20 to −30 degrees Celsius, less than about −30 degrees Celsius, e.g., −80 degrees Celsius, etc.) during the isolation process or after the isolation process).

In some embodiments, the aggrefatin protein has increased stability when isolated in a medium comprising glycerol, for example between about 20 to 30% glycerol, between about 25 to 35% glycerol, between about 30 to 40% glycerol, etc. In some embodiments, the aggrefatin protein has increased stability when isolated in a medium comprising a chelating agent (e.g., EDTA, ethylene glycol tetraacetic acid (EGTA), serum, oxalate, etc.).

The present invention also features a kit comprising an antibody specific for an isolated aggrefatin protein. The antibody may be a monoclonal antibody, however the present invention is not limited to a monoclonal antibody (e.g., the antibody may be a polyclonal antibody). In some embodiments, the antibody is raised to a sample obtained from a mammal (e.g., human, patient). The sample may have been stored at less than about −20 degrees Celsius. The sample may have been stored in a medium comprising ammonium sulfate.

The present invention also features a method of obtaining a measurement of an isolated aggrefatin protein. The method may comprise obtaining a sample (e.g., blood, CSF, etc.) from a mammal (e.g., human, patient); storing the sample in a medium that lacks a divalent cation chelating agent that chelates divalent cations at least as well as ethylenediaminetetraacetid acid (EDTA) such as citrate or heparin; and detecting a level of aggrefatin expression in the sample.

As used herein, a therapy or treatment may include but is not limited to glatiramer acetate (e.g., Copaxone®), natalizumab (e.g., Tysabri®), interferon beta-1a (e.g., Rebiff®, Avonex®), interferon beta-1b (e.g., Betaseron®), fingolimon (e.g., Gilenia®), cladribine, alemtuzumab (Campath®).

As used herein, the term “about” refers to plus or minus 10% of the referenced number. For example, an embodiment wherein the level of aggrefatin is about 20 fold higher includes an embodiment wherein the level of aggrefatin is between 18 to 22 fold higher.

### Example 1

The following example describes an assay for detecting the form of visfatin (e.g., a derivative thereof, a fragment thereof, an aggregate thereof, a visfatin complex, etc.), termed “aggrefatin.” The present invention is not limited to the examples described herein.

Briefly, an assay surface is first coated with a capture antibody. The capture antibody may be a monoclonal antibody that specifically binds to aggregated Visfatin. Next, a plasma or serum sample is added, and antigen present in the sample binds to the capture antibody. Horse Radish Peroxidase conjugated detecting monoclonal or polyclonal antibody is added (this can be any antibody that will bind to antigen). After this, substrate is added, which is converted by the enzyme to a detectable form.

The steps may be as follows: (1) Prepare a surface to which a known quantity of capture antibody is bound using any suitable method. Suitable methods include but are not limited to passive absorption or chemical coupling of capture antibody to the surface. (2) Block any available binding sites on the surface. (3) Apply the antigen-containing sample to the plate. (4) Wash the plate so that unbound antigen is removed. (5) Apply Horse Radish Peroxidase (HRP) conjugated detection antibodies, which also bind specifically to the antigen. (6) Wash the plate so that the unbound detection antibody is removed. (7) Apply a chromogenic substrate which is converted by the enzyme into a colored signal. (8) Measure the absorbency of the chromogenic signals of the plate wells to determine the presence and quantity of the antigen.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims.

1. A method of detecting a positive response to a therapy for a neurodegenerative disease or condition in a mammal, the method comprising:
(a) obtaining a first sample from the mammal and subjecting the first sample to a medium that lacks a divalent cation chelating agent or a medium that has a divalent cation chelating agent that does not chelate as many divalent cations as ethylenediaminetetraacetid acid (EDTA) does or to a medium comprising either heparin, a heparin derivative, a vitamin K agonist, an antithrombin activator, a direct thrombin inhibitor, or a combination thereof;
(b) administering the therapy to the mammal with the disease or condition;
(c) obtaining a second sample from the mammal and subjecting the second sample to a medium that lacks a divalent cation chelating agent or a medium that has a divalent cation chelating agent that does not chelate as many divalent cations as ethylenediaminetetraacetid acid (EDTA) does or to a medium comprising either heparin, a heparin derivative, a vitamin K agonist, an antithrombin activator, a direct thrombin inhibitor, or a combination thereof, the second sample is obtained a certain time period after the first sample;
(d) detecting a level of aggrefatin expression in both the first sample and the second sample; and
(e) comparing the level of aggrefatin expression in the first sample with a level of aggrefatin expression in the second sample, wherein a positive response to the therapy is detected if the level of the aggrefatin expression in the second sample is less than the level of the aggrefatin expression in the first sample.

2. The method of claim 1, wherein the therapy causes a change to a level of aggrefatin in the mammal.

3. The method of claim 1 further comprising obtaining a third sample from the mammal and subjecting the third sample to a medium that lacks a divalent cation chelating agent or a medium that has a divalent cation chelating agent that does not chelate as many divalent cations as ethylenediaminetetraacetid acid (EDTA) does or to a medium comprising either heparin, a heparin derivative, a vitamin K agonist, an antithrombin activator, a direct thrombin inhibitor, or a combination thereof, the third sample is obtained a certain time period after the second sample; detecting a level of aggrefatin expression the third sample; and comparing the level of aggrefatin expression in the third sample with either the level of aggrefatin in the first sample or the level of aggrefatin in the second sample, wherein a positive response to the therapy is detected if the level of the aggrefatin expression in the third sample is less than either the level of the aggrefatin expression in the first sample or the level of aggrefatin in the second sample.

4. The method of claim 1, wherein the medium that lacks a divalent cation chelating agent or the medium that has a divalent cation chelating agent that does not chelate as many divalent cations as ethylenediaminetetraacetid acid (EDTA) is citrate or oxalate.

5. The method of claim 1, wherein the first sample or the second sample is a blood sample or cerebrospinal fluid sample.

6. The method of claim 1, wherein detecting the level of aggrefatin expression comprises introducing an antibody to the sample, wherein the antibody binds to aggrefatin.

7. The method of claim 1, wherein detecting the level of aggrefatin expression comprises subjecting the sample to a western blot, an enzyme-linked immunosorbent assay (ELISA), a lateral flow assay, an immunohistochemistry assay, a radioimmunoassay, a bioluminescent assay, a chemiluminescent assay, a mass spectrometry assay, a flow cytometry assay, a fluorescent assay, a colorimetric assay, a enzymatic assay, a ligand binding assay, or a combination thereof.

8. The method of claim 1 further comprising detecting a level of a second biomarker in the sample.

9. The method of claim 8, wherein the second biomarker is perforin.

10. The method of claim 9, wherein detecting a level of perforin in the sample comprises detecting a percentage of CD16+/perforin+ cells.

11. The method of claim 9, wherein a positive response to the therapy is detected if the percentage of CD16+/perforin+ cells is more than about 75% of total CD16+ cells.

12. The method of claim 1, wherein the second biomarker is myelin basic protein (MPB) or a HLA DR2 related allele.

13. A method of detecting a positive response to a therapy for a stroke in a mammal, the method comprising:
(a) Obtaining a first sample from the mammal and subjecting the first sample to a medium that lacks a divalent cation chelating agent or a medium that has a divalent cation chelating agent that does not chelate as many divalent cations as ethylenediaminetetraacetid acid (EDTA) does;
(b) administering the therapy to the mammal with the stroke;
(c) obtaining a second sample from the mammal and subjecting the second sample to a medium that lacks a divalent cation chelating agent or a medium that has a divalent cation chelating agent that does not chelate as many divalent cations as ethylenediaminetetraacetid acid (EDTA) does, the second sample is obtained a certain time period after the first sample;
(d) detecting a level of aggrefatin expression in both the first sample and the second sample; and
(e) comparing the level of aggrefatin expression in the first sample with a level of aggrefatin expression in the second sample, wherein a positive response to the therapy is detected if the level of the aggrefatin expression in the second sample is less than the level of the aggrefatin expression in the first sample.

14. The method of claim 13, wherein the therapy causes a change to a level of aggrefatin in the mammal.

15. The method of claim 13, wherein the medium that lacks a divalent cation chelating agent or the medium that has a divalent cation chelating agent that does not chelate as many divalent cations as ethylenediaminetetraacetid acid (EDTA) is citrate or oxalate.

16. The method of claim 13, wherein the sample is a blood sample or cerebrospinal fluid sample.

17. The method of claim 13, wherein detecting the level of aggrefatin expression comprises introducing an antibody to the sample, wherein the antibody binds to aggrefatin.

18. The method of claim 13, wherein detecting the level of aggrefatin expression comprises subjecting the sample to a western blot, an enzyme-linked immunosorbent assay (ELISA), a lateral flow assay, an immunohistochemistry assay, a radioimmunoassay, a bioluminescent assay, a chemiluminescent assay, a mass spectrometry assay, a flow cytometry assay, a fluorescent assay, a colorimetric assay, a enzymatic assay, a ligand binding assay, or a combination thereof.

19. The method of claim 13 further comprising detecting a level of a second biomarker in the sample.

20. The method of claim 19, wherein the second biomarker is perforin, wherein detecting a level of perforin in the sample comprises detecting a percentage of CD16+/perforin+ cells.

21. The method of claim 20, wherein a positive response to the therapy is detected if the percentage of CD16+/perforin+ cells is more than about 75% of total CD16+ cells.

22. The method of claim 19, wherein the second biomarker is myelin basic protein (MPB) or a HLA DR2 related allele.

23. A method of detecting a neurodegenerative disease or condition in a mammal, the method comprising:
(a) obtaining a sample from the mammal;
(b) subjecting the sample to a medium that lacks a divalent cation chelating agent or a medium that has a divalent cation chelating agent that does not chelate as many divalent cations as ethylenediaminetetraacetid acid (EDTA) does or to a medium comprising either heparin, a heparin derivative, a vitamin K agonist, an antithrombin activator, a direct thrombin inhibitor, or a combination thereof; and
(c) detecting a level of aggrefatin expression in the sample;
wherein the neurodegenerative disease or condition is determined if the level of aggrefatin expression in the sample is more than about two standard deviations higher than an average level of aggrefatin expression in a control sample.

24. The method of claim 23, wherein the control sample is subjected to a medium that lacks a divalent cation chelating agent or a medium that has a divalent cation chelating agent that does not chelate as many divalent cations as ethylenediaminetetraacetid acid (EDTA) does or to a medium comprising either heparin, a heparin derivative, a vitamin K agonist, an antithrombin activator, a direct thrombin inhibitor, or a combination thereof.

25. The method of claim 23, wherein the medium that lacks a divalent cation chelating agent or the medium that has a divalent cation chelating agent that does not chelate as many divalent cations as ethylenediaminetetraacetid acid (EDTA) is citrate or oxalate.

26. The method of claim 23, wherein the sample is a blood sample or cerebrospinal fluid sample.

27. The method of claim 28, wherein detecting the level of aggrefatin expression comprises introducing an antibody to the sample, wherein the antibody binds to aggrefatin.

28. The method of claim 23, wherein detecting the level of aggrefatin expression comprises subjecting the sample to a western blot, an enzyme-linked immunosorbent assay (ELISA), a lateral flow assay, an immunohistochemistry assay, a radioimmunoassay, a bioluminescent assay, a chemiluminescent assay, a mass spectrometry assay, a flow cytometry assay, a fluorescent assay, a colorimetric assay, a enzymatic assay, a ligand binding assay, or a combination thereof.

29. The method of claim 23 further comprising detecting a level of a second biomarker in the sample.

30. The method of claim 29, wherein the second biomarker is perforin, wherein detecting a level of perforin in the sample comprises detecting a percentage of CD16+/perforin+ cells.

31. The method of claim 30, wherein the neurodegenerative disease or condition is detected if the percentage of CD16+/perforin+ cells is less than about 75% of total CD16+ cells.

32. The method of claim 29, wherein the second biomarker is myelin basic protein (MPB) or a HLA DR2 related allele.

33. The method of claim 23 further comprising administering a treatment to the mammal if the level of aggrefatin expression in the sample is more than about two standard deviations higher than an average level of aggrefatin expression in a control sample.

34. A method of monitoring disease activity of a neurodegenerative disease or condition in a mammal, the method comprising:
(a) obtaining a first sample from the mammal and subjecting the first sample to a medium that lacks a divalent cation chelating agent or a medium that has a divalent cation chelating agent that does not chelate as many divalent cations as ethylenediaminetetraacetid acid (EDTA) does or to a medium comprising either heparin, a heparin derivative, a vitamin K agonist, an antithrombin activator, a direct thrombin inhibitor, or a combination thereof;
(b) obtaining a second sample from the mammal and subjecting the second sample to a medium that lacks a divalent cation chelating agent or a medium that has a divalent cation chelating agent that does not chelate as many divalent cations as ethylenediaminetetraacetid acid (EDTA) does or to a medium comprising either heparin, a heparin derivative, a vitamin K agonist, an antithrombin activator, a direct thrombin inhibitor, or a combination thereof;
(c) detecting a level of aggrefatin expression in both the first sample and the second sample; and
(d) comparing the level of aggrefatin expression in the first sample with a level of aggrefatin expression in the second sample, wherein if:
(i) the level of the aggrefatin expression in the second sample is more than the level of the aggrefatin expression in the first sample then the disease activity has increased;
(ii) the level of the aggrefatin expression in the second sample is less than the level of the aggrefatin expression in the first sample then the disease activity has decreased;
(iii) the level of the aggrefatin expression in the second sample is about the same as the level of the aggrefatin expression in the first sample then the disease activity has not increased or decreased.

35. The isolated aggrefatin protein of claim 34, wherein the medium that lacks a divalent cation chelating agent or the medium that has a divalent cation chelating agent that does not chelate as many divalent cations as ethylenediaminetetraacetid acid (EDTA) is citrate or oxalate.

36. A method of monitoring, detecting, or predicting a subsequent stroke in a mammal, the method comprising:
(a) Obtaining a first sample from the mammal when the mammal has a stroke or after the mammal has had a stroke and subjecting the first sample to a medium that lacks a divalent cation chelating agent or a medium that has a divalent cation chelating agent that does not chelate as many divalent cations as ethylenediaminetetraacetid acid (EDTA) does or to a medium comprising either heparin, a heparin derivative, a vitamin K agonist, an antithrombin activator, a direct thrombin inhibitor, or a combination thereof;
(b) obtaining a second sample from the mammal and subjecting the second sample to a medium that lacks a divalent cation chelating agent or a medium that has a divalent cation chelating agent that does not chelate as many divalent cations as ethylenediaminetetraacetid acid (EDTA) does or to a medium comprising either heparin, a heparin derivative, a vitamin K agonist, an antithrombin activator, a direct thrombin inhibitor, or a combination thereof;
(c) detecting a level of aggrefatin expression in both the first sample and the second sample; and
(d) comparing the level of aggrefatin expression in the first sample with a level of aggrefatin expression in the second sample, wherein if the level of the aggrefatin expression in the second sample is more than the level of the aggrefatin expression in the first sample then the mammal is at high risk for a subsequent stroke.

37. The method of claim 36, wherein the medium that lacks a divalent cation chelating agent or the medium that has a divalent cation chelating agent that does not chelate as many divalent cations as ethylenediaminetetraacetid acid (EDTA) is citrate or oxalate.

38. The method of claim 36, wherein the sample is a blood sample or cerebrospinal fluid sample.

39. The method of claim 36, wherein detecting the level of aggrefatin expression comprises introducing an antibody to the sample, wherein the antibody binds to aggrefatin.

40. The method of claim 36, wherein detecting the level of aggrefatin expression comprises subjecting the sample to a western blot, an enzyme-linked immunosorbent assay (ELISA), a lateral flow assay, an immunohistochemistry assay, a radioimmunoassay, a bioluminescent assay, a chemiluminescent assay, a mass spectrometry assay, a flow cytometry assay, a fluorescent assay, a colorimetric assay, a enzymatic assay, a ligand binding assay, or a combination thereof.

41. The method of claim 36 further comprising detecting a level of a second biomarker in the sample.

42. The method of claim 41, wherein the second biomarker is perforin, wherein detecting a level of perforin in the sample comprises detecting a percentage of CD16+/perforin+ cells.

43. The method of claim 42, wherein the disease or condition is detected if the percentage of CD16+/perforin+ cells is less than about 75% of total CD16+ cells.

44. The method of claim 41, wherein the second biomarker is myelin basic protein (MPB) or a HLA DR2 related allele.

45. A kit comprising an antibody specific for an isolated aggrefatin protein, the isolated aggrefatin protein comprising an epitope of Phe-Lys-Asp-Pro-Val-Ala-Asp-Pro-Asn-Lys-Arg (SEQ ID NO 1) and having a molecular weight between about 200 kDa to 800 kDa.

46. The kit of claim 45, wherein the antibody is a monoclonal antibody.

47. The kit of claim 45, wherein the antibody is raised to a sample obtained from a mammal, the sample having been stored at less than about −20 degrees Celsius in a medium comprising ammonium sulfate.

48. An isolated aggrefatin protein comprising an epitope of Phe-Lys-Asp-Pro-Val-Ala-Asp-Pro-Asn-Lys-Arg (SEQ ID NO 1), the isolated aggrefatin protein having a molecular weight between about 200 kDa to 800 kDa.

49. The isolated aggrefatin protein of claim 48, wherein the isolated aggrefatin protein is a homogeneous aggregate of visfatin.

50. The isolated aggrefatin protein of claim 48, wherein the isolated aggrefatin protein is a heterogeneous aggregate of visfatin.

51. The isolated aggrefatin protein of claim 48, wherein the isolated aggrefatin protein is isolated in citrate, heparin, oxalate, a heparin derivative, a vitamin K agonist, an antithrombin activator, a direct thrombin inhibitor, or a combination thereof.

52. The isolated aggrefatin protein of claim 48, wherein the isolated aggrefatin protein has increased stability when isolated in a medium comprising glycerol.

53. The isolated aggrefatin protein of claim 52, wherein the medium comprises between about 25 to 35% glycerol.

54. The isolated aggrefatin protein of claim 48, wherein the isolated aggrefatin protein has increased stability when isolated in a medium comprising a chelating agent.

55. The isolated aggrefatin protein of claim 54, wherein the chelating agent is ethylenediaminetetraacetic acid.

56. The isolated aggrefatin protein of claim 54, wherein the chelating agent is ethylene glycol tetraacetic acid.

57. The isolated aggrefatin protein of claim 48, wherein the isolated aggrefatin protein has increased stability when isolated in serum.

58. A method of obtaining a measurement of an isolated aggrefatin protein, said method comprising:
(a) obtaining a sample from a mammal;
(b) storing the sample in a medium that lacks a divalent cation chelating agent or a medium that has a divalent cation chelating agent that does not chelate as many divalent cations as ethylenediaminetetraacetid acid (EDTA) does or in a medium comprising either heparin, a heparin derivative, a vitamin K agonist, an antithrombin activator, a direct thrombin inhibitor, or a combination thereof; and
(c) detecting a level of aggrefatin expression in the sample.

59. The method of claim 58, wherein the medium that lacks a divalent cation chelating agent or the medium that has a divalent cation chelating agent that does not chelate as many divalent cations as EDTA does is citrate or oxalate.

60. The method of claim 58, wherein the sample is a blood sample or cerebrospinal fluid sample.

61. A method of modifying a sample obtained from a mammal, said method comprising subjecting the sample to either (i) a medium comprising glycerol; (ii) a medium that lacks a divalent cation chelating agent; (iii) a medium that has a divalent cation chelating agent that does not chelate as many divalent cations as ethylenediaminetetraacetid acid (EDTA) does; (iv) a medium comprising either heparin, a heparin derivative, a vitamin K agonist, an antithrombin activator, a direct thrombin inhibitor, or a combination thereof; or (v) a medium comprising ammonium sulfate.

62. The method of claim 61, wherein the medium comprising glycerol comprises between about 25 to 35% glycerol.

63. The method of claim 61, wherein the medium that lacks a divalent cation chelating agent or the medium that has a divalent cation chelating agent that does not chelate as many divalent cations as EDTA does is citrate or oxalate.

64. The method of claim 61, wherein the medium comprising ammonium sulfate comprises between about 5 to 30% saturated ammonium sulfate.


SEQUENCE LISTING (3 sequences)

SEQ ID NO: 1 - protein, 11 aa, Homo sapiens
FKDPVADPNKR

SEQ ID NO: 2 - protein, 28 aa, Homo sapiens
CYWLTNWIETILVQSWYPITVATNSREQ

SEQ ID NO: 3 - protein, 25 aa, Homo sapiens
VTKSYSFDEIRKNAQLNIELEAAHH